(19) United States
(12) Patent Application Publication
(10) Pub. No.: US 2025/0099567 A1
(43) Pub. Date: Mar. 27, 2025

(54) METHOD FOR INDUSTRIAL PRODUCTION OF VACCINE AGAINST PSEUDOMONAS AERUGINOSA

(71) Applicant: WESTVAC BIOPHARMA CO., LTD., Chengdu, Sichuan (CN)

(72) Inventors: Zhenling WANG, Chengdu (CN); Yuquan WEI, Chengdu (CN)

(73) Assignee: WESTVAC BIOPHARMA CO., LTD., Chengdu (CN)

(21) Appl. No.: 18/833,626

(22) PCT Filed: Feb. 21, 2022
(86) PCT No.: PCT/CN2022/076983

§ 371 Date: Jul. 26, 2024

(30) Foreign Application Priority Data
Jan. 29, 2022 (CN) 202210110275.5

(51) Int. Cl. A61K 39/104; C12N 1/02; C12N 1/20; C12N 13/00; C12R 1/385

(52) U.S. Cl. CPC A61K 39/104; C12N 1/02; C12N 1/20; C12N 13/00; C12R 2001/385

(57) ABSTRACT

A method for industrial production of vaccine against Pseudomonas aeruginosa adopts a series of standardized, programmed, and digitized settings to produce a vaccine with stable quality comprising whole bacterial cells and various internal immunogenic components of bacterial cell. The obtained vaccine has good immunogenicity, and can prevent various infectious diseases caused by Pseudomonas aeruginosa with low side effects and high safety.

### TECHNICAL FIELD

The invention belongs to the field of bioengineering, and in particular relates to a method for industrial production of vaccine against Pseudomonas aeruginosa.

### BACKGROUND

Pseudomonas aeruginosa, also known as green pus bacillus, is a common opportunistic pathogen with strong resistance to various external environments and inactivation measures. It can survive for extended periods, particularly in humid environments. This bacterium is widely found in many places such as soil, water, air, human and animal skin, mucous membrane, intestinal tract, and upper respiratory tract. Its pathogenic characteristics often result in secondary infections, primarily affecting patients with weakened immune systems, such as those with extensive burns, long-term use of immunosuppressants, cancer, or hypoproteinemia. Infections caused by Pseudomonas aeruginosa can occur in any part of the human body. Clinically, common infections include skin and subcutaneous tissue infections, including otitis media, meningitis, respiratory tract infections, urinary tract infections, and sepsis. Surveys in recent years report that over 30% of nosocomial infections are caused by Pseudomonas aeruginosa. Currently, infections caused by Pseudomonas aeruginosa are mainly treated with drugs. However, the overuse of antibiotics in recent years has led to the emergence of numerous antibiotic-resistant strains, exacerbating the threat posed by Pseudomonas aeruginosa to patients. The well-established mechanisms of Pseudomonas aeruginosa resistance to antibiotics include: 1. The outer membrane permeability of Pseudomonas aeruginosa is reduced, which prevents the antibiotics from entering the bacterial inner membrane target; 2. Pseudomonas aeruginosa cells expel a wide range of structurally unrelated antibiotics or other toxic substances out of the cell through active efflux; 3. Pseudomonas aeruginosa change the quantity or structure of penicillin-binding proteins (PBPs), the target of antibiotics, to reduce the affinity of the drug for PBPs, thereby leading to intrinsic resistance; 4. Plasmid or chromosome-mediated B-lactamase inactivates the antibiotics. Therefore, the current Pseudomonas aeruginosa infection has reached the stage where no antibiotics are ineffective, and it is the general trend to prevent and control Pseudomonas aeruginosa infection through immunization.

A vaccine is a substance that can stimulate the body's immune system and activate T and B lymphocytes to produce antibodies or immune cells specific to target antigens (viruses, bacteria, etc.). At present, the preparation methods of Pseudomonas aeruginosa vaccines studied all over the world have involved multiple technical approaches, including heat- or formaldehyde-treated inactivated vaccines, attenuated vaccines, subunit vaccines, and single-component vaccines (such as pilus, flagella, outer membrane proteins OprI/OprF). In basic experiments, single-component vaccines have shown good preventive effects, but due to their limited antigen components, they can only target infections of specific diseases (such as pneumonia). Inactivated (by heat, formaldehyde, etc.) or attenuated (by gene mutation) whole-cell vaccines offer a broader spectrum of protection. However, due to the inactivation of some unknown antigen components and reduced bacterial metabolic activity during the preparation process, these vaccines often have poorer immunogenicity and preventive effects. Additionally, incomplete inactivation can leave significant residual toxicity in the vaccines. The development of whole-cell vaccines has been attempted for various bacterial species (such as Shigella, Neisseria gonorrhoeae, Neisseria meningitidis), but most of these vaccines have failed, indicating the difficulty of producing effective whole-cell vaccines. Moreover, the current preparation process for Pseudomonas aeruginosa whole-cell vaccines still relies primarily on traditional formaldehyde inactivation. Formaldehyde is classified as a Group 1 carcinogen by the WHO, and individuals allergic to formaldehyde cannot use these vaccines, highlighting the urgent need for new inactivation methods.

However, the method disclosed in the prior art for producing Pseudomonas aeruginosa whole-cell vaccines is only suitable for small scale production, and is not suitable for large scale and industrialized production. Based on this, the present invention provides a method for industrial production of multi-component and whole-cell Pseudomonas aeruginosa vaccines, which can alleviate one or more of the existing problems.

### SUMMARY

The invention provides a method for industrial production of vaccine against Pseudomonas aeruginosa, and a vaccine against Pseudomonas aeruginosa prepared by the method, and the specific technical solution is as follows.

A method for industrial production of vaccine against Pseudomonas aeruginosa, comprising the following steps:
S1 culturing a Pseudomonas aeruginosa strain for production with a suitable culture medium to prepare a seed liquid; the culture medium should not comprise animal-derived components and is not limited to the culture medium involved in the embodiments of the present invention.
S2 inoculating the seed liquid into a fermentation tank at 2%˜10% of fermentation volume to perform fermentation, wherein fermentation temperature is 30˜40° C., pH value is 5˜9, rotation speed is 100˜400 rpm, aeration rate is 2˜5 L/min, dissolved oxygen is 10%˜30%, and fermentation time is 3˜8 h;
S3 monitoring a bacterial cell density in the fermentation tank, taking a bacterial solution from the fermentation tank when the bacterial cell density in the fermentation tank reaches a standard, directly centrifuging the bacterial solution at a centrifugal force of 3000˜8000×g for 10˜30 min to collect the bacterial cell;
S4 resuspending the bacterial cell with an isotonic injection and adjusting a concentration of the bacterial cell, and then irradiating the bacterial cell to cause the bacterial cell to lose proliferative activity, wherein irradiation rays of the irradiating comprises one or more of X-rays, γ-rays, and rays generated by an isotope radiation source Co⁶⁰, the isotonic injection comprises physiological saline and the like;
S5 taking the bacterial solution after irradiating for examination, the bacterial solution comprises whole bacterial cell and internal immunogenic component of bacterial cell; when a proportion of the whole bacterial cell is more than 80%, the bacterial solution after irradiating is qualified;
S6 resuspending the bacterial solution qualified in the step S5 with the isotonic injection and adjusting a concentration of the bacterial solution qualified to 1.0×10⁷˜3.0×10⁷ bacteria/mL to prepare the vaccine against Pseudomonas aeruginosa.

Further, the bacterial cell density in the fermentation tank reaches a standard when an absorbance value of the bacterial cell density reaches 1˜3 OD.

Further, the internal immunogenic component of bacterial cell comprises membrane vesicles, nucleic acids, and bacterial fragments.

Further, the step S4 further comprises a step of determining composition and content of the bacterial solution after irradiating, and a method for the determining composition and content of the bacterial solution after irradiating comprises one or more of spectrophotometry, density gradient centrifugation, scanning electron microscopy, and transmission electron microscopy.

Further, a basic inactivation dose for the irradiating is not less than 1000 Gy, and a total dose for the irradiating is greater than the basic inactivation dose for the irradiating.

Further, a manner of the irradiating is low dose rate, long time, and continuous irradiation; the dose rate is preferably 5˜15 Gy/min, and time for the continuous irradiation is preferably greater than 2 h.

Further, a step of determining an inactivation time or an inactivation dose for the irradiating comprises:
a) determining a material and shape of container for an irradiation sample; the material is a medical-grade plastic material, which comprises but is not limited to polypropylene (PP), polystyrene (PS), etc.; the shape is preferably column, barrel or bag;
b) determining a maximum loading volume for inactivation;
c) determining a concentration of the bacterial solution: determining a total amount of an inactivated bacterial cell in each batch according to production design, and then selecting an appropriate concentration of the bacterial solution according to loading requirement, a calculation formula of the concentration of the bacterial solution is:

$$N ( bacteria / mL ) = \frac{A ( dose ) \times B ( bacteria / dose )}{V ( mL )} ,$$

wherein N represents the concentration of the bacterial solution, A represents the total number of vaccine dose to be produced, B represents a bacterial dosage per vaccine dose, V represents a total volume of the bacterial solution, and V≤the maximum loading volume;
d) selecting a dose rate: designing different dose rate groups, performing inactivation of the maximum loading volume according to the steps a)˜c) with the time set for the continuous irradiation to be no less than 2 h; specifically, it can be set to 2 h or any time greater than or equal to 2 h;
e) determining a sterilization curve: performing multiple batches of irradiation according to the dose rate and the time for the irradiation determined in the step d); and performing viable bacteria counting at an intermediate time point or an extended time point, or performing viable bacteria counting at an intermediate irradiation dose point or an extended irradiation dose point, to determine the sterilization curve;
f) determining the inactivation time or the inactivation dose: according to the sterilization curve, taking adjacent first time point, second time point, and third time point, or taking adjacent first irradiation dose point, second irradiation dose point, and third irradiation dose point to perform viable bacteria counting; wherein an interval between the time points is not less than 20 min, and an interval between the irradiation dose points is not less than 200 Gy; when the counting results are less than 100 CFU/mL, 0 CFU/mL, and 0 CFU/mL, respectively, the second time point is the inactivation time, and the second irradiation dose point is the inactivation dose.

Further, after preliminarily determining the inactivation time or the inactivation dose, a key parameter for further optimization comprises dose rate, irradiation time, loading volume, bacterial concentration, etc., and may also comprise inactivation dose; the above parameters can be adjusted within an appropriate range in order to adapt to industrialized production.

Further, after the determining an inactivation time or an inactivation dose, the method further comprises inactivation verification, and the inactivation verification comprises sterility testing and stability testing after the inactivation; the stability testing comprises accelerated testing at 25° C. or 37° C. and real-time testing at 2˜8° C. The purpose of this step is to ensure that Pseudomonas aeruginosa in the vaccine will not proliferate in the long-term storage process.

Further, industrial production verification is carried out. After selecting an appropriate industrial production parameter, multiple batches of production verification should be carried out to ensure that each batch can achieve complete inactivation of bacteria.

A vaccine against Pseudomonas aeruginosa prepared by the method for industrial production provided by the present invention, a dosage form of the vaccine is preferably injection and/or freeze-dried powder, and a vaccination method of the vaccine optionally comprises one or more of subcutaneous injection, intramuscular injection, skin scarification, nasal administration, and oral administration. The vaccine is a whole bacterial cell vaccine, or a multi-component vaccine, which comprises not only whole bacterial cells, but also internal immunogenic components of bacterial cell such as membrane vesicles, nucleic acids, and bacterial fragments.

Use of the vaccine against Pseudomonas aeruginosa prepared by the present invention in the preparation of medicament for preventing an infectious disease caused by Pseudomonas aeruginosa, and the infectious disease may typically comprise Pseudomonas aeruginosa pneumonia, chronic obstructive pulmonary disease combined with Pseudomonas aeruginosa infection, burn combined with Pseudomonas aeruginosa infection, Pseudomonas aeruginosa keratitis, etc.

### Term Definition

The “basic inactivation dose” mentioned in the present invention refers to the dose that can basically inactivate the bacterial cell, with the lower limit being 1000 Gy.

The “total dose” mentioned in the present invention refers to the cumulative irradiation dose over time during irradiation process.

The “inactivation dose” mentioned in the present invention refers to the irradiation dose (also referred to as “the most optimum inactivation dose”) at which all bacterial cells in the inactivated bacterial solution (also referred to as “inactivated stock solution”) are just inactivated. “Just inactivated” means that all bacterial cells in the inactivated solution reach an irreversible state of death. In some embodiments of the invention, based on the plotted sterilization curve, three adjacent irradiation dose points are selected for viable bacteria counting. If the counting results are less than 100 CFU/mL, 0 CFU/mL, and 0 CFU/mL, respectively, the intermediate irradiation dose point among the three adjacent irradiation dose points is the inactivation dose. The interval between the irradiation dose points should be equal and no less than 200 Gy. Further, for example, when the dose is at the “inactivation dose−200 Gy” condition, the number of viable bacteria comprised in the inactivation stock solution is less than 100 CFU/mL; at the inactivation dose, the number of the viable bacteria comprised in the inactivation stock solution is 0 CFU/mL; and at the “inactivation dose+200 Gy” condition, the number of the viable bacteria comprised in the inactivation stock solution is 0 CFU/mL. Similarly, in some embodiments, based on the plotted sterilization curve, three adjacent time points are selected for viable bacteria counting. If the counting results are less than 100 CFU/mL, 0 CFU/mL, and 0 CFU/mL, respectively, the intermediate time point among the three adjacent time points is the inactivation time. The interval between time points should be equal and no less than 20 min. Further, for example, when the time is at the “inactivation time−20 min” condition, the number of viable bacteria comprised in the inactivation stock solution is less than 100 CFU/mL; at the inactivation time, the number of viable bacteria comprised in the inactivation stock solution is 0 CFU/mL; and at the “inactivation time+20 min” condition, the number of viable bacteria comprised in the inactivation stock solution is 0 CFU/mL.

When compared with the prior art, the present invention has the following beneficial effects:
(1) The present invention provides a method for industrial production of vaccine against Pseudomonas aeruginosa. This method adopts a series of standardized, programmed, and digitized settings to produce a vaccine with stable quality comprising whole bacterial cells and various internal immunogenic components of bacterial cell. Specifically, the bacterial solution with bacterial cell density reaching the standard is directly centrifuged in the present invention, skipping the purification step of repetitive centrifugation and washing. This increases industrial production efficiency and prevents the rupture of bacterial cells during large-scale production, which could release harmful substances like endotoxins. Therefore, in the industrial production process, repetitive centrifugation and washing will increase the safety risk of the prepared vaccine. A large number of bacterial cell ruptures caused by repetitive centrifugation and washing could decrease the number of whole bacterial cells and increase cell debris, which is removed by centrifugation and washing. In addition, the purification step of repetitive centrifugation and washing will also lead to a reduction in the immunogenic components of vaccine prepared by the present invention, such as membrane vesicles and nucleic acids, thereby leading to unstable quality of the industrially produced vaccines and reduced variety and quantity of immunogenic components. Based on this, direct centrifugation in the industrial production method provided by the present invention avoids the above-mentioned risks caused by the purification step, and improves the effectiveness and safety of the vaccine.
(2) The present invention optimizes the irradiation manner of irradiating, that is, adopts low dose rate (5˜15 Gy/min), long time, continuous irradiation (greater than 2 h). This reduces the total dose of irradiating (≤2000 Gy), thereby preventing damage to the bacterial cells by large dose of irradiation rays and improving the vaccine's immunogenicity and safety.
(3) The vaccine prepared by the present invention has good immunogenicity, not only the actual inoculation dosage is low, but also can prevent various infectious diseases caused by Pseudomonas aeruginosa, such as Pseudomonas aeruginosa pneumonia, chronic obstructive pulmonary disease combined with Pseudomonas aeruginosa infection, burn combined with Pseudomonas aeruginosa infection, Pseudomonas aeruginosa keratitis, etc. The vaccine prepared by the present invention may typically be adjuvant-free, i.e., in the vaccine prepared by the present invention, no adjuvant is required to enhance the immune response of the body. Certainly, in some scenarios (for example, there is a need to prepare a highly immunogenic Pseudomonas aeruginosa vaccine), the vaccine prepared by the present invention may also be combined with an adjuvant. The realization of the above-mentioned good immunogenicity (protective efficacy of the vaccine) not only benefits from the optimization of the preparation method as described above, but also the optimization of the actual immunization procedure of the vaccine: while reducing the total times of vaccination, the immunization interval is prolonged. The present invention finds that the optimized immunization procedure is beneficial to improve the actual efficacy of the vaccine, and prolonging the immunization interval is more in line with the body's immune response to the vaccine.
(4) The technical solution of the present invention adopts a physical method (i.e. irradiation rays) for inactivation. Compared with conventional chemical methods (for example, using formaldehyde as a chemical inactivator), the present invention has no chemical (chemical inactivator) residue, avoiding allergic reactions and carcinogenic risks caused by a chemical inactivator to a certain extent, enhancing the safety of the vaccine, and minimizing side effects.

### DESCRIPTION OF DRAWINGS

To make the embodiments of the present invention or the technical solutions in the prior art clearer, the drawings required to be used in the description of the embodiments or the prior art will be briefly introduced below. It is obvious that the drawings described below are some embodiments of the present invention, and that other drawings can be obtained from these drawings for those of ordinary skill in the art without making inventive effort.

FIG. 1 shows the protective effect of the vaccine prepared by the method provided by the present invention in the mouse Pseudomonas aeruginosa pneumonia model;

FIG. 2 shows the protective effect of the vaccine prepared by the method provided by the present invention in COPD combined with Pseudomonas aeruginosa infection model;

FIG. 3 shows the protective effect of the vaccine prepared by the method provided by the present invention in the mouse burn model;

FIG. 4 shows the protective effect of the vaccine prepared by the method provided by the present invention in the mouse corneal infection model;

FIG. 5 shows a schematic diagram of the sterilization curve.

### DETAILED DESCRIPTION

To make the objective, the technical solutions and advantages of the embodiments of the present invention clearer, the technical solutions of the embodiments of the present invention will be clearly and completely described below in combination with drawings. It is obvious that the described embodiments are some of the embodiments of the present invention, not all of the embodiments. Based on the embodiments of the present invention, all other embodiments obtained by those of ordinary skill in the art without making inventive effort shall belong to the protection scope of the present invention.

It should be noted that the term “include”, “comprise” or any variant thereof is intended to encompass nonexclusive inclusion so that a process, method, article or device including a series of elements includes not only those elements but also other elements which have been not listed definitely or an element(s) inherent to the process, method, article or device. Moreover, the expression “comprising a(n) . . . ” in which an element is defined will not preclude presence of an additional identical element(s) in a process, method, article or device comprising the defined element(s) unless further defined.

As used herein, the term “about”, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

### Embodiment 1: A Preparation Method of Pseudomonas aeruginosa Vaccine (Vaccine Against Pseudomonas aeruginosa)

S1 culturing a Pseudomonas aeruginosa strain for production with a suitable culture medium to prepare a seed liquid; wherein the culture medium should not comprise animal-derived components, for example, Tryptone Soy Broth (TSB).

S2 inoculating the seed liquid into a fermentation tank at 2%˜10% of fermentation volume to perform fermentation, wherein fermentation temperature is 30˜40° C., pH value is 5˜9 (preferably 7), rotation speed is 100˜400 rpm, aeration rate is 2˜5 L/min, dissolved oxygen is 10%˜30%, and fermentation time is 3˜8 h;

S3 monitoring a bacterial cell density in the fermentation tank, taking a bacterial solution from the fermentation tank when the bacterial cell density in the fermentation tank reaches a standard (that is, absorbance of the bacterial cell density reaches 1˜3 OD), directly centrifuging the bacterial solution at a centrifugal force of 3000˜8000×g for 10˜30 min to collect the bacterial cell;

S4 resuspending the bacterial cell with an isotonic injection and adjusting a concentration of the bacterial cell, and then irradiating the bacterial cell with irradiation rays to cause the bacterial cell to lose proliferative activity, wherein irradiation rays of the irradiating comprises one or more of X-rays, y-rays, and rays generated by an isotope radiation source Co⁶⁰; the isotonic injection comprises physiological saline and the like. The method further comprises a step of determining composition and content of the bacterial solution after irradiating. One or more of spectrophotometry, density gradient centrifugation, scanning electron microscopy, and transmission electron microscopy may be used for the determining composition and content of the bacterial solution.

S5 taking the bacterial solution after irradiating for examination, the bacterial solution comprises whole bacterial cell and internal immunogenic component of bacterial cell; when a proportion of the whole bacterial cell is more than 80%, the bacterial solution after irradiating is qualified; the internal immunogenic component of bacterial cell comprises membrane vesicles, nucleic acids, and bacterial fragments. Under normal circumstances, the proportion of the whole bacterial cell in the bacterial solution obtained by the method for industrial production provided by the present invention should be kept at more than 80%. The purpose of examining the bacterial solution is to ensure that the quantity of the component (whole bacterial cell and internal immunogenic component) of the bacterial solution remains stable under the situation of adjusting other parameters.

S6 resuspending the bacterial solution qualified in the step S5 with the isotonic injection and adjusting a concentration of the bacterial solution qualified to 1.0×10⁷˜3.0×10⁷ bacteria/mL to prepare the vaccine against Pseudomonas aeruginosa.

Further, a basic inactivation dose for the irradiating is not less than 1000 Gy, and a total dose for the irradiating is greater than the basic inactivation dose for the irradiating. A manner of the irradiating is low dose rate, long time, continuous irradiation. The dose rate for the irradiating is 5˜15 Gy/min, and time of the continuous irradiation is greater than 2 h. In some embodiments, the total dose for the irradiating in the present invention can be controlled within the range of ≤2000 Gy.

A step of determining inactivation time or inactivation dose for the irradiating comprises:
a. Determining a material and shape of container for an irradiation sample. The material is a medical-grade plastic material, which comprises but is not limited to polypropylene (PP), polystyrene (PS), etc. The shape is preferably one or more of column, barrel or bag.
b. Determining a maximum loading volume for inactivation. The loading volume is determined by the shape of the container and the height of the liquid level. The difference between the dose rate at the highest (nearest) liquid level and the lowest (farthest) liquid level should not exceed 20% of the dose rate at the highest (nearest) liquid level. The volume of the bacterial solution corresponding to the highest (nearest) liquid level is the maximum loading volume for inactivation. To measure this, a radiation dose detector is used. The detector probes are placed at the highest (nearest) liquid level and the lowest (farthest) liquid level relative to the radiation source. When the irradiation device is activated, the radiation dose detector will read the irradiation dose rate. The 20% threshold is based on quality control requirements to ensure the uniformity of the irradiation dose received by the irradiation sample.
c. Determining a concentration of the bacterial solution. Determining a total amount of an inactivated bacterial cell in each batch according to production design, and then selecting an appropriate concentration of the bacterial solution according to loading requirement, a calculation formula of the concentration of the bacterial solution is:

$$N ( bacteria / mL ) = \frac{A ( dose ) \times B ( bacteria / dose )}{V ( mL )} ,$$

wherein N represents the concentration of the bacterial solution, A represents the total number of vaccine dose to be produced, B represents a bacterial dosage per vaccine dose, V represents a total volume of the bacterial solution, and V≤the maximum loading volume.
d. Selecting a dose rate. Designing different dose rate groups, performing inactivation of the maximum loading volume according to the steps a˜c with the time set for the continuous irradiation to be no less than 2 h; specifically, it can be set to 2 h or any time greater than or equal to 2 h.
e. Determining a sterilization curve. Performing multiple batches of irradiation according to the dose rate and the time for the irradiation determined in the step d; and performing viable bacteria counting at an intermediate time point or an extended time point, or performing viable bacteria counting at an intermediate irradiation dose point or an extended irradiation dose point, to determine the sterilization curve. It should be noted that the inactivation process can be controlled by adjusting either the irradiation time or the irradiation dose. Consequently, the inactivation parameter can be examined based on time control or dose control. For example, after determining the inactivation time, samples can be taken at time points before (e.g., “intermediate time point”) and after (e.g., “extended time point”) the determined inactivation time. This is to verify whether the inactivation time meets the requirement for complete inactivation. Similarly, after determining the inactivation dose, samples can be taken at irradiation dose points before (e.g., “intermediate irradiation dose point”) and after (e.g., “extended irradiation dose point”) the determined inactivation dose. This is to verify whether the inactivation dose meets the requirement for complete inactivation.
f. Determining the inactivation time or the inactivation dose. According to the sterilization curve, taking adjacent three time points (first time point, second time point, and third time point), or adjacent three irradiation dose points (first irradiation dose point, second irradiation dose point, and third irradiation dose point) to perform viable bacteria counting; wherein an interval between the time points is not less than 20 min, and an interval between the irradiation dose points is not less than 200 Gy; when the counting results are less than 100 CFU/mL, 0 CFU/mL, and 0 CFU/mL, respectively, the intermediate time point (the second time point) is the inactivation time, and the intermediate irradiation dose point (the second irradiation dose point) is the inactivation dose. A schematic diagram of the sterilization curve is shown in FIG. 5. According to the sterilization curve, the samples at adjacent three irradiation dose points “1200 Gy”, “1400 Gy” and “1600 Gy” are taken to perform viable bacteria counting. When the counting results are less than 100 CFU/mL, 0 CFU/mL, and 0 CFU/mL respectively, the irradiation dose point “1400 Gy”, the intermediate irradiation dose point among the three adjacent irradiation dose points, is the inactivation dose. Similarly, according to the plotted sterilization curve, the samples at three adjacent time points “120 min”, “140 min” and “160 min” are taken to perform viable bacteria counting. When the counting results are less than 100 CFU/mL, 0 CFU/mL, and 0 CFU/mL respectively, the time point “140 min”, the intermediate time point among the three adjacent time points, is the inactivation time.

After preliminarily determining the inactivation time or the inactivation dose, a key parameter for further optimization comprises dose rate, irradiation time, loading volume, bacterial concentration, etc., and may also comprise inactivation dose; the above parameters can be adjusted within an appropriate range in order to adapt to industrialized production.

After the determining an inactivation time or an inactivation dose, the method further comprises inactivation verification, and the inactivation verification comprises sterility testing and stability testing after the inactivation; the stability testing comprises accelerated testing at 25° C. or 37° C. and real-time testing at 2˜8° C. The purpose of this step is to ensure that Pseudomonas aeruginosa in the vaccine will not proliferate in the long-term storage process.

Industrial production verification is carried out. After selecting an appropriate industrial production parameter, multiple batches of production verification should be carried out to ensure that each batch can achieve complete inactivation of bacteria.

A vaccine against Pseudomonas aeruginosa prepared by the method for industrial production provided by the present invention, the vaccine is a whole bacterial cell vaccine, or a multi-component vaccine, which comprises not only whole bacterial cell, but also internal immunogenic components of bacterial cell such as membrane vesicles, nucleic acids, and bacterial fragments. A dosage form of the vaccine is injection and/or freeze-dried powder, and a vaccination method of the vaccine optionally comprises one or more of subcutaneous injection, intramuscular injection, skin scarification, nasal administration, and oral administration.

In some embodiments of the present invention, a mass ratio of whole bacterial cell, membrane vesicles, nucleic acids, and bacteria fragments is preferably 98:1:0.1:1. Under the condition of this ratio, the vaccine has better immunogenicity and less side effects.

### Embodiment 2: The Protective Effect of the Vaccine in the Mouse Pseudomonas aeruginosa Pneumonia Model

There are 20 serotypes of Pseudomonas aeruginosa, and the mainstream serotypes of Pseudomonas aeruginosa isolated clinically are O6, O11, and O4, accounting for about 20%, 15%, and 10%, respectively. In this embodiment, Pseudomonas aeruginosa PA1 (serotype O5) is used to prepare a vaccine according to the above process. According to the 0, 14 and 28-day immunization procedure, the mice are subcutaneously immunized with 0.5 mL of vaccine comprising 1.0×10⁷ bacteria. 7 days after the last immunization, the airways of mice are infected with Pseudomonas aeruginosa PA1 (O5), clinically isolated carbapenem-resistant Pseudomonas aeruginosa C58 (O6), standard strains ATCC33358 (O11), ATCC33351 (O4), and laboratory strain PA14 (O14). 24 hours after infection, lung tissues are taken to count the bacterial load. The results are shown in FIG. 1. The vaccine can effectively reduce the bacterial load of PA1 (O5) (P<0.001), C58 (O6) (P<0.05), ATCC33358 (O11) (P<0.01), ATCC33351 (O4) (P>0.05), PA14 (P<0.01). The results show that the vaccine have a protective effect on the infection of different serotypes of Pseudomonas aeruginosa, and could prevent various infections caused by Pseudomonas aeruginosa.

### Embodiment 3: The Protective Effect of the Vaccine in Mouse Chronic Obstructive Pulmonary Disease (COPD) Combined with Pseudomonas aeruginosa Infection Model

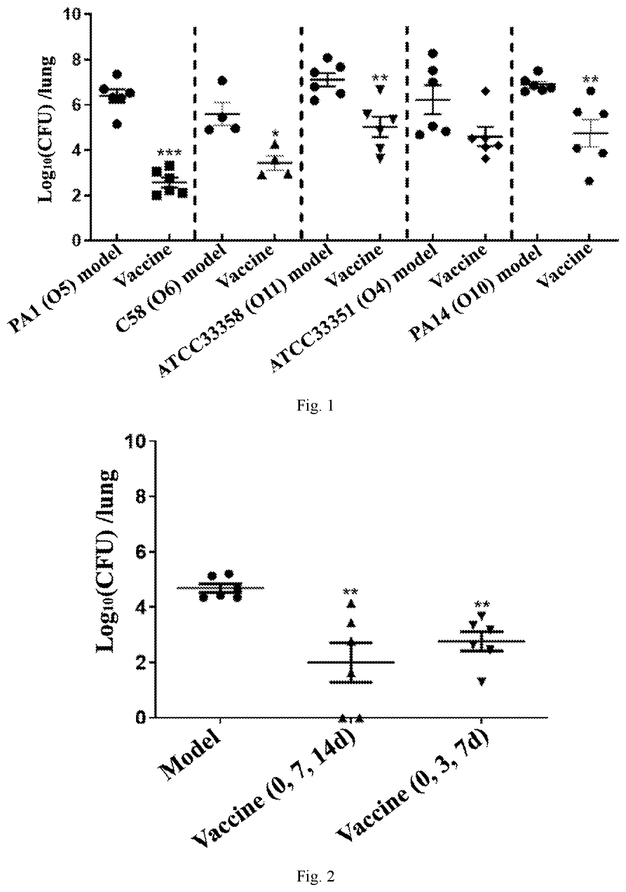

In this embodiment, Pseudomonas aeruginosa PA1 (serotype O5) is used to prepare a vaccine according to the above process. According to two immunization procedures (0, 3, 7 d and 0, 7, 14 d), the mice are subcutaneously immunized with 0.5 mL of vaccine comprising 1.0×10⁷ bacteria. The COPD model is established by infusion of elastase 10 days before the infection. 7 days after the last immunization, the mice are infected with the same bacterial strain (Pseudomonas aeruginosa PA1) through the airways of the lungs. The mice are sacrificed 24 hours after infection, and the lung tissues are taken to count the bacterial load. The results are shown in FIG. 2. Immunization of the vaccine according to two procedures has obvious in vivo protective effect on COPD combined with Pseudomonas aeruginosa pulmonary infection (P<0.01), and can lead to about 2 log₁₀ reduction in the bacterial load of lung tissues (clearing 99% of bacteria). The results show that the vaccine had a significant protective effect on COPD combined with Pseudomonas aeruginosa pulmonary infection.

### Embodiment 4: The Protective Effect of the Vaccine in the Mouse Burn Model

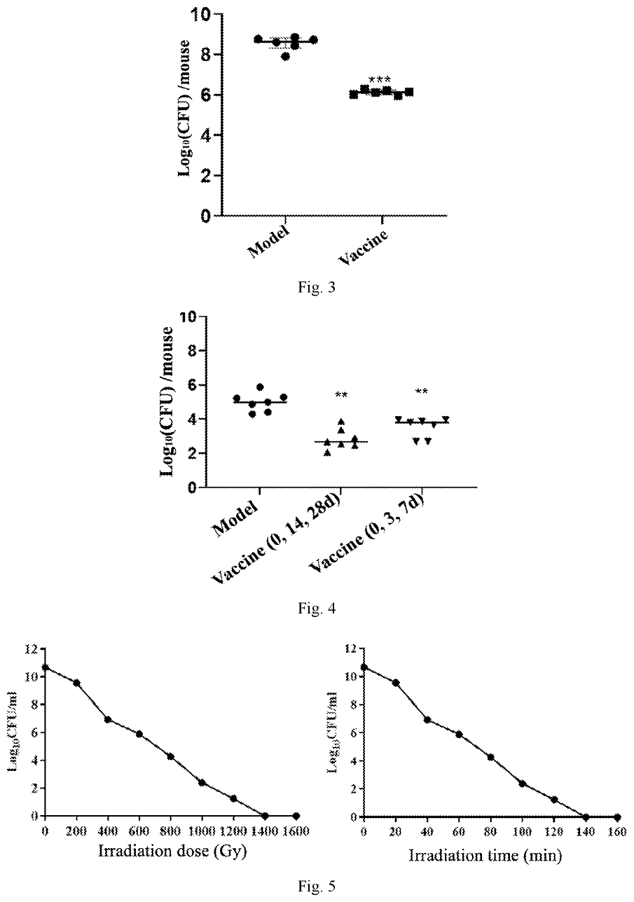

In this embodiment, Pseudomonas aeruginosa PA1 (serotype O5) is used to prepare a vaccine according to the above process. According to the 0, 14 and 28-day immunization procedure, the mice are subcutaneously immunized with 0.5 mL of vaccine comprising 1.0×10⁷ bacteria. 7 days after the last immunization, the dorsal skin of the mice is scalded with a hot air blower to establish a burn model. The wound is subcutaneously infected with Pseudomonas aeruginosa PA1 2 h after burn and the skin of the wound is taken 24 hours after burn to count the bacterial load. The results are shown in FIG. 3. Vaccine immunization can significantly reduce the bacterial load of burn wounds combined with Pseudomonas aeruginosa infection (P<0.001). The results show that the vaccine has a significant protective effect on burns combined with Pseudomonas aeruginosa infection.

### Embodiment 5: The Protective Effect of the Vaccine in the Mouse Corneal Infection Model

In this embodiment, Pseudomonas aeruginosa PA1 (serotype O5) is used to prepare a vaccine according to the above process. According to two immunization procedures (0, 3, 7 d and 0, 7, 14 d), the mice are immunized with nasal drops (20 μL of vaccine, comprising 1.0×10⁷ bacteria). 7 days after the last immunization, the cornea of the mouse is infected with the same bacterial strain (Pseudomonas aeruginosa PA1) by scratching the cornea using a needle tip. The results are shown in FIG. 4. Immunization with the vaccine according to two procedures has obvious protective effect on Pseudomonas aeruginosa corneal infection (P<0.01). The results show that the vaccine had obvious protective effect on corneal infection by Pseudomonas aeruginosa.

The embodiments of the present invention are described above with reference to the accompanying drawings, but the present invention is not limited to the aforementioned specific embodiments. The aforementioned embodiments are merely illustrative and not limiting. For those of ordinary skill in the art, many forms can be made under the teaching of present invention without departing from the spirit of the present invention and the scope of the claims, all of which shall fall within the protection scope of the present invention.

1. A method for industrial production of vaccine against Pseudomonas aeruginosa, wherein comprising the following steps:
S1 culturing a Pseudomonas aeruginosa strain for production with a suitable culture medium to prepare a seed liquid;
S2 inoculating the seed liquid into a fermentation tank at 2%˜10% of fermentation volume to perform fermentation, wherein fermentation temperature is 30˜40° C., pH value is 5˜9, rotation speed is 100˜400 rpm, aeration rate is 2˜5 L/min, dissolved oxygen is 10%˜30%, and fermentation time is 3˜8 h;
S3 monitoring a bacterial cell density in the fermentation tank, taking a bacterial solution from the fermentation tank when the bacterial cell density in the fermentation tank reaches a standard, directly centrifuging the bacterial solution at a centrifugal force of 3000˜8000×g for 10˜30 min to collect the bacterial cell;
S4 resuspending the bacterial cell with an isotonic injection and adjusting a concentration of the bacterial cell, and then irradiating the bacterial cell to cause the bacterial cell to lose proliferative activity, wherein irradiation rays of the irradiating comprises one or more of X-rays, γ-rays, and rays generated by an isotope radiation source Co⁶⁰;
S5 taking the bacterial solution after irradiating for examination, the bacterial solution comprises whole bacterial cell and internal immunogenic component of bacterial cell; when a proportion of the whole bacterial cell is more than 80%, the bacterial solution after irradiating is qualified;
S6 resuspending the bacterial solution qualified in the step S5 with the isotonic injection and adjusting a concentration of the bacterial solution qualified to 1.0×10⁷˜3.0×10⁷ bacteria/mL to prepare the vaccine against Pseudomonas aeruginosa.

2. The method according to claim 1, wherein the bacterial cell density in the fermentation tank reaches a standard when an absorbance value of the bacterial cell density reaches 1˜3 OD.

3. The method according to claim 1, wherein the internal immunogenic component of bacterial cell comprises membrane vesicles, nucleic acids, and bacterial fragments.

4. The method according to claim 1, wherein the step S4 further comprises a step of determining composition and content of the bacterial solution after irradiating, and a method for the determining composition and content of the bacterial solution after irradiating comprises one or more of spectrophotometry, density gradient centrifugation, scanning electron microscopy, and transmission electron microscopy.

5. The method according to claim 1, wherein a basic inactivation dose for the irradiating is not less than 1000 Gy, and a total dose for the irradiating is greater than the basic inactivation dose for the irradiating.

6. The method according to claim 5, wherein a manner of the irradiating is low dose rate, long time, and continuous irradiation; the dose rate is preferably 5˜15 Gy/min, and time for the continuous irradiation is preferably greater than 2 h.

7. The method according to claim 1, wherein a step of determining an inactivation time or an inactivation dose for the irradiating comprises:
a) determining a material and shape of container for an irradiation sample;
b) determining a maximum loading volume for inactivation;
c) determining a concentration of the bacterial solution: determining a total amount of an inactivated bacterial cell in each batch according to production design, and then selecting an appropriate concentration of the bacterial solution according to loading requirement, a calculation formula of the concentration of the bacterial solution is:
$$N ( bacteria / mL ) = \frac{A ( dose ) \times B ( bacteria / dose )}{V ( mL )} ,$$
wherein N represents the concentration of the bacterial solution, A represents the total number of vaccine dose to be produced, B represents a bacterial dosage per vaccine dose, V represents a total volume of the bacterial solution, and V≤the maximum loading volume;
d) selecting a dose rate: designing different dose rate groups, performing inactivation of the maximum loading volume according to the steps a)˜c) with the time set for the continuous irradiation to be no less than 2 h;
e) determining a sterilization curve: performing multiple batches of irradiation according to the dose rate and the time for the irradiation determined in the step d); and performing viable bacteria counting at an intermediate time point or an extended time point, or performing viable bacteria counting at an intermediate irradiation dose point or an extended irradiation dose point, to determine the sterilization curve;
f) determining the inactivation time or the inactivation dose: according to the sterilization curve, taking adjacent first time point, second time point, and third time point, or taking adjacent first irradiation dose point, second irradiation dose point, and third irradiation dose point to perform viable bacteria counting; wherein an interval between the time points is not less than 20 min, and an interval between the irradiation dose points is not less than 200 Gy; when the counting results are less than 100 CFU/mL, 0 CFU/mL, and 0 CFU/mL, respectively, the second time point is the inactivation time, and the second irradiation dose point is the inactivation dose.

8. The method according to claim 7, wherein after the determining an inactivation time or an inactivation dose, the method further comprises inactivation verification, and the inactivation verification comprises sterility testing and stability testing after the inactivation; the stability testing comprises accelerated testing at 25° C. or 37° C. and real-time testing at 2˜8° C.

9. (canceled)

10. (canceled)
